(19) United States
(12) Patent Application Publication
(10) Pub. No.: US 2022/0071254 A1
(43) Pub. Date: Mar. 10, 2022

(54) CONCENTRATE ADDITION SYSTEM

(71) Applicant: Radient Technologies Innovations Inc., Edmonton (CA)

(72) Inventors: Michael Cabigon, Edmonton (CA); Jim Seethram, Edmonton (CA); Steven Splinter, Edmonton (CA); Denis Taschuk, Edmonton (CA)

(21) Appl. No.: 17/237,801

(22) Filed: Apr. 22, 2021

Related U.S. Application Data
(63) Continuation of application No. PCT/IB2019/058782, filed on Oct. 15, 2019.
(60) Provisional application No. 62/749,077, filed on Oct. 22, 2018.

(51) Int. Cl. A23L 33/105; G16C 20/90

(52) U.S. Cl. CPC A23L 33/105; G16C 20/90

(57) ABSTRACT

Methods and systems of adding a consistent amount of cannabis concentrate to various food products are disclosed. The methods and systems include calculating the quantity of cannabis concentrate permitted in the various food products, determining a permitted variability in the quantity of cannabis concentrate for each of the food products, transmitting the quantity of cannabis concentrate and permitted variability to a manufacturing system, applying a cannabis concentrate to a food product using an application device, evaluating the amount of cannabis concentrate actually applied to the food product, and flagging the food products that exceed the permitted variability.

### CROSS-REFERENCE TO RELATED APPLICATION

The present patent application is a continuation of International Application No. PCT/IB2019/058782 filed Oct. 15, 2019, which claims the priority benefit of U.S. provisional patent application No. 62/749,077 filed Oct. 22, 2018, the disclosures of which are incorporated by reference herein.

### BACKGROUND OF THE INVENTION

### 1. Field of the Disclosure

The present disclosure is generally related to methods and systems for manufacturing edible cannabis products. More specifically, the present disclosure relates to methods and systems for the mass manufacturing edible cannabis products having a consistent cannabinoid dosage in each edible product.

### 2. Description of Related Art

Cannabis is a genus belonging to the family cannabaceae. There are three common species of cannabis including Cannabis stavia, Cannabis indica, and Cannabis ruderalis. The genus cannabaceae is indigenous to Central Asia and the Indian subcontinent and has a long history of being used for medicinal, therapeutic, and recreational purposes. For example, cannabis is known to be capable of relieving nausea (such as that accompanying chemotherapy), pain, vomiting, spasticity in multiple sclerosis, and increase hunger in anorexia.

Cannabis contains a unique class of terpeno-phenolic compounds known as cannabinoids, or phytocannabinoids. The principle cannabinoids present in a cannabis can include Delta-9-tetrahydrocannabinolic acid (THCA) and cannabidiolic acid (CBDA). THCA does not include psychoactive properties on it's own, but when decarboxylated THCA becomes Delta-9-tetrahydrocannabinol (THC), which is a potent psychoactive cannabinoid. CBDA can be decarboxylated into cannabidiol (CBD), which is a major cannabinoid substituent in hemp cannabis. CBD is a non-psychoactive cannabinoid and is widely known to have therapeutic potential for a variety of medical conditions including, but not limited to, those described above.

Historical delivery methods of cannabinoids have included combustion (such as smoking) of the dried cannabis plant material, or biomass. However, smoking can result in adverse effects on a user's respiratory system due to the production of potentially toxic substances. Moreover, smoking is an inefficient mechanism which delivers a variable mixture of both active and inactive substances, many of which may be undesirable. Common alternative delivery methods, including but not limited to, ingestion, typically require an extraction process to be performed on the cannabis biomass to remove the desired components. Such ingestible cannabis items can include, but are not limited to, concentrates, extractants, and cannabis oils.

A cannabis edible, also known as a cannabis-infused food, edible cannabis product, or simply an “edible,” is a food product which contains one or more cannabinoids, as described above. Although the term “edible” may refer to either a food or a drink, a cannabis-infused drink may be referred to as a liquid edible or “drinkable.” For the purposes of this invention, “food product” can encompass any form of cannabis edible including liquid edibles. Most edibles contain a significant amount of THC, which can induce a wide range of effects, including, but not limited to, relaxation, euphoria, increased appetite, fatigue, and anxiety. THC-dominant edibles can be consumed for both recreational and medical purposes. In the alternative, some edibles can only contain a negligible amount of THC, and intended to provide other cannabinoids, most commonly cannabidiol (CBD). Such CBD edibles are primarily used for medical purposes. Foods and beverages made from such non-psychoactive cannabis products are sometimes known as hemp foods.

Food products containing cannabis extract (edibles) have emerged as a popular and lucrative facet of the legalized cannabis market for both recreational and medicinal uses. However, the many formulations of cannabis extracts used in such edibles present a unique regulatory challenge for policy makers. Though edibles are often considered a safe, discreet, and effective means of attaining the desired therapeutic and/or intoxicating effects of cannabis without exposure to the potentially harmful risks of smoking, there has been little research into how ingestion of cannabinoids differs from other methods of cannabis administration in terms of therapeutic efficacy, subjective effects, and safety. The most prominent difference between ingestion and inhalation of a cannabis extract is the delayed onset of the cannabinoid effect with ingestion. For example, consumers often do not understand this aspect of edible use and have been found to consume a greater than intended amount of cannabinoid before the cannabinoids have taken effect, often resulting in profoundly adverse effects.

Due to the potential of accidental overdose, regulatory bodies have started to impose limits on the quantity of cannabinoids which can be present in a cannabis edible product, including limits on the percent variability of the quantity of cannabinoids.

The present disclosure relates to methods and systems for accurate integration of extracted cannabinoids into food and beverage products during the manufacturing process. The systems and methods described herein are operable to standardize procedures which integrate cannabinoids into food processing for edible manufacturers, thus ensuring consistent dosage across both a population of edible products and across the area of each individual product.

### SUMMARY OF THE CLAIMED INVENTION

Examples of the present disclosure provide systems and methods for providing consistent amounts of cannabis concentrate to a food product in a manufacturing system. In particular, a method for calculating the amount of cannabis concentrate including calculating the quantity of cannabis concentrate permitted in the various food products, determining a permitted variability in the quantity of cannabis concentrate for each of the food products, transmitting the quantity of cannabis concentrate and permitted variability to a manufacturing system, applying a cannabis concentrate to a food product using an application device, evaluating the amount of cannabis concentrate actually applied to the food product, and flagging the food products that exceed the permitted variability.

In addition to providing consistent amounts of cannabis concentrate into food products, the methods and systems described herein can be used to remove food products which exceed the permitted variability. Such systems and methods can assist manufacturers in abiding with local regulations regarding the amount of cannabinoids allowed in edible cannabis products.

### BRIEF DESCRIPTIONS OF THE DRAWINGS

FIG. 1 illustrates an exemplary network environment in which a system for controlling consistent amounts of cannabis-based concentrates added to food products may be implemented.

FIG. 2 is a flowchart illustrating an exemplary method for analytics-based control of cannabis-based concentrates added to food products.

FIG. 3 is a flowchart illustrating an exemplary method for transmitting control data from the analytics module to the manufacturing system via a communication network.

FIG. 4 is a flowchart illustrating an exemplary method for performing a quality control check during manufacture of food products that include cannabis-based concentrates.

### DETAILED DESCRIPTION

Because cannabis does not naturally include THC, but rather its precursor THCA, cannabis must be decarboxylated in order to convert THCA into THC. THCA may degrade into THC, which may then degrade into cannabinol over time. THCA can be rapidly, albeit not completely in many instances, decarboxylated when heated. Comparing the effects of eating cannabis products and smoking them is difficult and subject to large margins of error due to wide variability in how different people smoke, the number, duration, and spacing of puffs, the hold time, and the volume of the person's lungs, all of which may result in different types and extent of effects of the smoked dosage.

With regard to eating, the different vehicles in which cannabinoids are dissolved for oral consumption can affect the availability of the cannabinoids to be absorbed. Additionally, different people can metabolize the same products differently. Generally, however, because oral cannabis doses are processed by the digestive system and the liver before entering the bloodstream, ingested cannabinoids may be absorbed more slowly, have delayed and lower peak concentrations, and be cleared through the user's system more slowly in comparison to the inhaling the same amount of cannabinoids in an aerosol (e.g, which can be formed when cannabis is burnt for smoking).

Oral administration of cannabinoids generally leads to two concentration peaks, due to enterohepatic circulation. Consuming THC through ingestion results in absorption through the liver and, through metabolic processes, the conversion of a significant proportion of it into 11-hydroxy-THC, which is more potent than THC and crosses the blood-brain barrier more easily.

Due to the potential for overdose, as described above, many governments have instituted regulations on the amount of cannabinoids which can be added to edibles. For example, Health Canada has imposed a limit of 10 mg of THC per discrete unit of edible cannabis product and per immediate container. Specifically, to meet the regulation requirements, in a container containing one discrete unit (such as a cookie or gummy), that unit may contain up to 10 mg of THC. In the alternative, in a container having two discrete units, each unit can contain up to 5 mg of THC. Moreover, according to the regulation, if the total quantity of a cannabinoid, THC or CBD, that is displayed on the label exceeds 5 mg, the product is subject to a 15% variability limit; if the quantity of cannabinoid is more than 2 mg but less than 5 mg, the variability limit is 20%, and if the quantity of cannabinoid is less than 2 mg, the variability limit will be 25%. Limits, such as these aim to address the key public health risks associated with edible cannabis, including, but not limited to, the risks of overconsumption and accidental consumption.

Examples of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

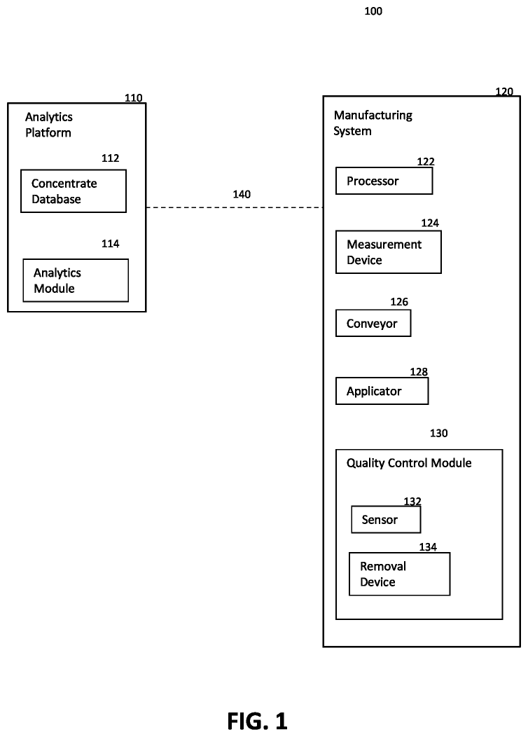

FIG. 1 illustrates an exemplary network environment in which a system 100 for controlling consistent amounts of cannabis-based concentrates added to food products may be implemented. The present disclosure provides a system and method for adding consistent quantities of cannabinoid concentrate to food products. A system in accordance with the present disclosure will be described with reference to FIG. 1. The system 100 can include at least an analytics platform server 110 and a manufacturing system 120 coupled to and communicable with one another via a communication network 140. The analytics platform server 110 is operable to store a combination of food product data and quantity and potency (such as concentration of cannabinoids) of additive cannabinoids in a cannabis concentrate in a concentrate database 112. Specifically, the concentrate database 112 can store information relating to legal regulations limiting the amount of different, and total, cannabinoids allowable in an edible cannabis product. The concentration database 112 can also store information relating to various food products, specifically with respect to the quantity of cannabis concentrate necessary to be added to said food products to achieve desired consumer effect or quantity of cannabinoids.

The analytics platform server 110 is also operable to determine, via an analytics module 114, the quantity of cannabis concentrate desired based on the type of food product to be produced, and the desired quantity, or dosage, of cannabinoids allowed in the final food product. The analytics platform server 110 can then transmit the quantity of cannabis concentrate, or dosage, to the manufacturing system 120 via the communication network 140. The analytics module 114 can be a software program operable to determine the optimal quantity of cannabis concentrate to add to the manufacturing process of a food product and send the determined quantity to the manufacturing system to ensure consistent quantity across the product line.

The communication network 140 may be a wired and/or a wireless network. The communication network, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art. The communication network can allow ubiquitous access to shared pools of configurable system resources and higher-level services that can be rapidly provisioned with minimal management effort, often over Internet and relies on sharing of resources to achieve coherence and economies of scale, like a public utility, while third-party clouds enable organizations to focus on their core businesses instead of expending resources on computer infrastructure and maintenance. The modules, databases, and networks described with respect to FIG. 1 can be stored on, and accessible via, the cloud.

The manufacturing system 120 is operable to manufacture edible cannabis products including adding a consistent amount of cannabis concentrate to each food product that is produced by the system. The manufacturing system described herein can be any known system for manufacturing a food product. The system 100 described with respect to FIG. 1 can be operable for manufacturing systems 120 making both large and small batch productions. The manufacturing system 120 can include a measurement device 124, a conveyor 126, an applicator 128, and a quality control module 130. The manufacturing system 120 can manufacture products by moving them via the conveyor 126, applying a cannabis concentrate using an applicator 128, and screening each food product after the application of the cannabis concentrate with a sensor 132 of the quality control module 130. The quality control module 130 can then remove the food items that do not meet the standard using a removal device 134. In at least one example, the manufacturing system 100 can include a processor 122 having a memory operable to store instructions thereon. The processor 122 can be operable to receive the quantity data of the cannabis concentrate from the analytics module 114 of the analytics platform server 110 and sends the quantity data to each of the measurement device 124 and the Quality Control module 130.

Specifically, the measurement device 124 can be any device operable to determine a consistent amount of cannabis concentrate to add to the product based on the quantity received from the analytics module 114. In at least one example, the measurement device can include a weighing scale, a volume measurement apparatus, or any other suitable measurement device. The conveyor 126 of the manufacturing system 120 can be any device on which the food products can be placed for the application of a cannabis concentrate. An applicator 128 operable to apply a cannabis concentrate to a food product in the appropriate form. For example, the cannabis concentrate can be provided to the manufacturing system 120 in various forms including, but not limited to, liquid, powdered, resin, or crystalized form. In an alternative example, the cannabis concentrate can be added as part of a food product ingredient. Such ingredients can include, but are not limited to, butters, oils, and combinations thereof. Based on the food product being produced, the manufacturing system can apply the ideal form of cannabis concentrate. Additionally, the applicator 128 can be selected based on the form of cannabis concentrate to be added. For example, the applicator 128 can include a sprayer device for coating the food products and a mixing device to ensure that the consistent quantity of cannabis concentrate is spread evenly throughout the food product.

Once the cannabis concentrate is added to the food product, the conveyor 126 can move the food product to a quality control module 130. The quality control module 130 can be operable to determine the amount of cannabis concentrate added by the applicator. For example, the quality control module 130 can include a sensor 132, such as an optical sensor, operable to detect the amount of cannabis concentrate. The sensor 132 can then send the information to quality control module 130 to determine whether the amount of cannabis concentrate detected exceeds a predetermined threshold. In at least one example, the sensor 132 can be a hyperspectral camera and the quality control module can be operable to perform a hyperspectral analysis on the image. In an alternative embodiment, the sensor can be any other known cannabinoid testing method including, but not limited to, high performance liquid chromatography (HPLC), gas chromatography-mass spectrometry (GCMS), liquid chromatography-mass spectrometry (LCMS), and the like. The quality control module 130 can include a software that is operable to receive information on the quantity of cannabis concentrate for each item as it passes by the sensor 132, determine the acceptable error range of cannabis concentrate based on the information received from the analytics module 114, and flag the items whose cannabis concentrate quantity exceeds the acceptable error range. The quality control module 130 can further include a removal device 134, which can be a mechanism operable to remove the flagged food product items before the products proceed to a packaging stage.

Table 1, below, illustrates an exemplary entry from the concentrate database 112, as described with respect to FIG. 1. As shown in Table 1, the concentrate database can include information relating to the number of servings of a food product made per batch. The concentration database can also include information relating to legal regulations including, but not limited to the quantity of cannabinoids allowed in each food product and the allowable variability limit. When cannabis concentrates are created, each concentrate can be provided an identification number and tested for potency, such information can be stored in the concentrate database as well. The information stored in the concentrate database can be transmitted from the analytics platform to the quality control module of the manufacturing system via a communication network as described above. It should be understood that the information in Table 1 is merely exemplary, and should not be construed as limiting.

TABLE 1

| | Quantity of | | | | Cannabis Concentrate | | | Quantity of | |
|---|---|---|---|---|---|---|---|---|---|
| No. of | Cannabinoids | | Cannabis | Cannabis | Potency (% w/w) | | | Cannabis | Permitted |
| Servings | in Food | Variability | Concentrate | Concentrate | | | Total | Concentrate | Variability |
| per Batch | Product (mg) | Limit | Type | ID | THC | CBD | Cannabinoids | (g) | (+/−g) |
| 100,000 | 5 | 15% | Liquid | C00002 | 50 | 0 | 50 | 1,000.00 | 150.00 |
| 100,000 | 10 | 15% | Liquid | C00025 | 0 | 65 | 65 | 1,538.46 | 230.77 |
| 250,000 | 2 | 20% | Liquid | C00200 | 70 | 0 | 70 | 714.29 | 142.86 |
| 500,000 | 5 | 15% | Powder | C01200 | 0 | 95 | 95 | 2,631.58 | 394.74 |

In an alternative example, the concentrate database can include information relating to the food product type, food product name, food product ID, serving size, number of servings produced per production batch, the quantity, or dosage, of cannabinoids in each food product, variability limit of cannabinoids allowed in the food product, cannabis concentrate type, cannabis concentrate ID, potency of cannabis concentrate, quantity of cannabis concentrate available for use, and permitted variability in cannabis concentrate potency. In at least one example, the food product type may be baked goods, the food product name may be chocolate chip cookie, the food product ID may be F00001, the serving size may be 15 g per discrete unit, the number of servings per production batch may be 100,000, the quantity, or dosage, of cannabinoids in food product may be 5 mg, the variability limit of cannabinoids in the food product may be ±15%, the cannabis concentrate type may be liquid extract, the cannabis concentrate ID may be C00002, the potency of the cannabis concentrate may be 50% w/w THC. Then, the quantity of cannabis concentrate, or the amount of cannabis concentrate to be added to the production batch of 100,000 chocolate chip cookies, may be 1000 g and the permitted variability, or the variability in the amount of cannabis concentrate to be added to the production batch of 100,000 chocolate chip cookies, may be ±150 g.

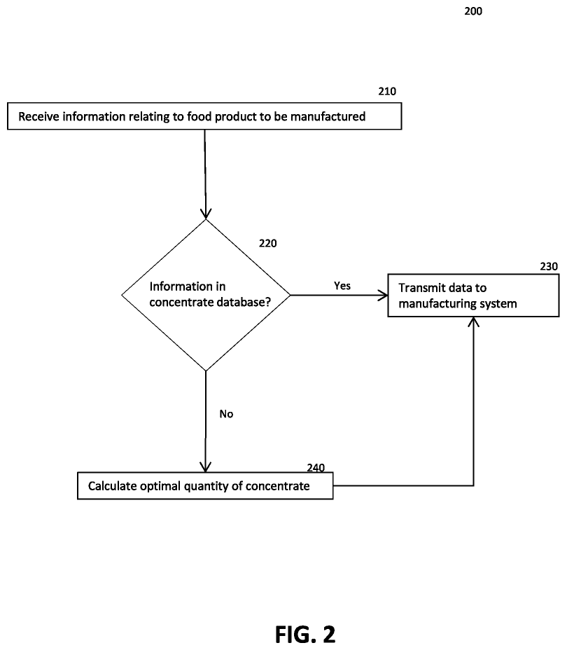

The functioning of the analytics module is explained with reference to FIG. 2. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

FIG. 2 is a flowchart illustrating an exemplary method 200 for analytics-based control of cannabis-based concentrates added to food products. Specifically, FIG. 2 illustrates a method 200 performed via execution of the analytics module. The method 200 can begin at block 210 by receiving information relating to the product to be manufactured. The information can include, at least, food product type, food product name, food product identification number, and the serving size of the final product. At block 220, a search of the concentrate database for pre-existing entries can be performed. Such entries can include, but are not limited to, the cannabis concentrate type and cannabis concentrate ID previously used if the same food product has been previously manufactured. The entries can further include the quantity of cannabis concentrate and permitted variability allowable for the manufacturing of a production batch of the food product. If previous entries are found, the method 200 can proceed to block 230, where the data relating to the food product and the cannabis concentrate can be transmitted to the manufacturing system.

In the alternative, if no previous entry is found, the analytics module can calculate the optimal quantity of cannabis concentrate for the food product. The analytics module can then retrieve the cannabis variability permitted by the relevant regulations and can store the quantity of cannabinoids per discrete unit and permitted variability for the desired food product in the concentrate database. The quantity of cannabis concentrate may be calculated using one of several methods, which can include, for example, multiplying the quantity of cannabinoids allowable in each food product (mg) by the number of servings per production batch, then dividing by the cannabis concentrate potency (% w/w), as shown in equation (1).

$$\frac{\begin{matrix}( Cannabinoids per discrete unit ) \times \\ ( Servings per Batch )\end{matrix}}{Potency} \qquad ( 1 )$$

The permitted variability of cannabis concentration may be calculated by multiplying the quantity of cannabis concentrate (g) by the variability limit of cannabinoids in each food product (%), as shown in Equation (2).

(Cannabis Concentrate Quantity)×(Variability Limit) (2)

In some examples, the target quantity of cannabinoids in a food product and variability limit of cannabinoids in the food product may be imposed by regulation or market demands. In the alternative, the permitted variability may be calculated based on an ideal variability, or error range, for each dose (quantity of cannabinoid) divided by the serving size. In at least one example the ideal error range may be +/−2 mg for an individual dose. As such, if the serving size is 5 pieces, the error range may be +/−0.4 mg per individual piece.

Once the optimal quantity of cannabis concentrate has been calculated at block 240, the method 200 can proceed to block 230, where the quantity of cannabis concentrate and permitted variability are transmitted to the manufacturing system.

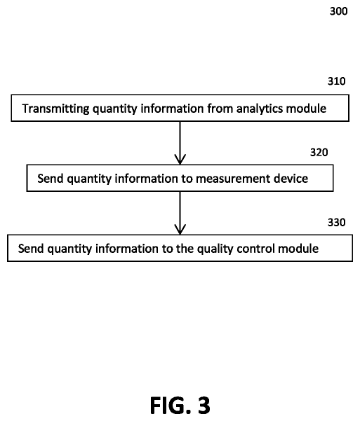

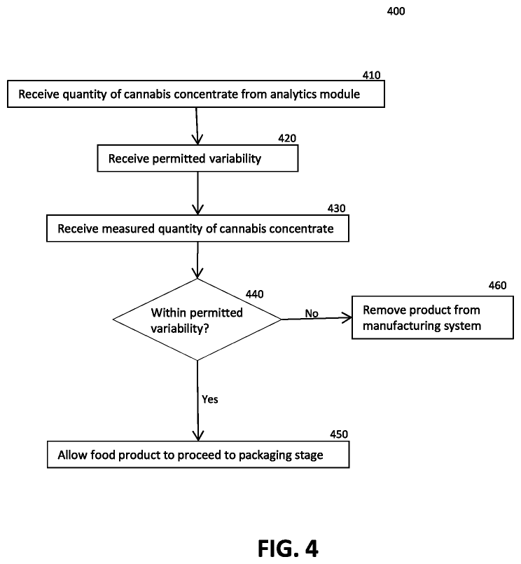

FIG. 3 is a flowchart illustrating an exemplary method 300 for transmitting control data from the analytics module to the manufacturing system via a communication network. The functioning of the communication network is explained with reference to FIG. 3. An exemplary method 300 for transmitting information from an analytics platform server 110 to a manufacturing system 120 is illustrated in FIG. 3. The method 300 can begin at block 310 where information relating to the quantity of cannabis concentrate and permitted variability associated with the food product, as determined in method 200, are transmitted from the analytics module to the manufacturing system. At block 320, the quantity of cannabis concentrate calculated above is transmitted, via the communication network, to the measurement device. As described above, the measurement device can be operable to measure the appropriate quantity of cannabis concentrate, as indicated by the analytics module, to the food products during the manufacturing process. Once the cannabis concentrate is added to the food products, the food products can be sent to a quality control module. At block 330, the quantity of cannabis concentrate for each of the food products is sent to the quality control module. The functioning of the quality control module is explained with reference to FIG. 4, below.

FIG. 4 is a flowchart illustrating an exemplary method for performing a quality control check during manufacture of food products that include cannabis-based concentrates. Such method 400 may result in quality control checks on the quantity of cannabis concentrate in the manufactured food product. The method 400 can begin at block 410, where the quantity of cannabis concentrate associated with the food product is received from the analytics module, as described above. At block 420, the quality control module can receive an input relating to the permitted variability associated with the food product from the analytics module. At block 430, the measured quantity of the cannabis concentrate for each of the food product items is received from the measurement device.

At block 440, the sensor of the quality control module measures the amount of cannabis concentrate in each of the final food items. In at least one example, as described above, the sensor can be an optical sensor, such as a hyperspectral camera. The quality control module can be operable to analyze the information received from the optical sensor using hyperspectral analysis. The quality control module can then compare the amount of cannabis concentrate in a final composition of each of the food items with the permitted variability received from the analytics module. If the quantity of cannabis concentrate is within the permitted variability, the method 400 can proceed to block 450, where the food product is allowed to proceed throughout the manufacturing process to a packaging stage. In the alternative, if the quantity of cannabis concentrate is determined to exceed the permitted variability, the method 400 can proceed to block 460, where the food items are flagged for removal. The removal device of the quality control module, as described with respect to FIG. 1, can then be actuated to remove the flagged items from the manufacturing system.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claims.

What is claimed is:

1. A method for adding cannabis-based concentrates to food products, the system comprising:
storing information in a database in memory regarding a plurality of different food products, the stored information regarding each food product including a limit on cannabis-based additives;
receiving a request for information regarding a specified food product, the request sent from a manufacturing system over a communication network to an analytics platform server;
identifying a quantity of at least one cannabis-based additive that falls within the limit associated with the specified food product based on the stored information;
transmitting the identified quantity of the at least one cannabis-based additive from the analytics module to the manufacturing system via the communications network, wherein the manufacturing system is instructed to add the identified quantity of cannabis-based additive to the food product; and
evaluating a final composition of the food product to confirm an amount of the cannabis concentrate in the final composition of the food product.

2. The method of claim 1, wherein the limit on cannabis-based additives includes a permitted range of variability.

3. The method of claim 1, wherein identifying the quantity of the at least one cannabis-based additive includes retrieving the stored information regarding the specified food product from the database, and applying the associated limit to a serving size of the specified food product.

4. The method of claim 1, wherein the stored information regarding to the specified food product includes at least one of serving size, a number of servings per production batch, and a type of food product.

5. The method of claim 1, wherein the request specifies at least one of serving size and number of servings of the specified food product.

6. The method of claim 1, wherein the manufacturing system is instructed to add the cannabis-based additive to the specified food product by:
measuring out the identified quantity of the at least one cannabis-based additive using a measurement device; and
applying the measured quantity of cannabis-based additive to the specified food product via an applicator.

7. The method of claim 1, wherein evaluating a final composition of the food product further comprises:
measuring the amount of the cannabis-based additive that is detected in the final composition of the food product using a sensor;
comparing the measured amount of detected cannabis-based additive to the identified quantity; and
determining that the comparison between the measured amount and the identified quantity falls within the limit associated with the specified food product.

8. The method of claim 1, further comprising removing the food product from a production line of the manufacturing system via a removal device based on the amount of cannabis-based additive exceeding the limit associated with the specified food product.

9. The method of claim 1, further comprising selecting a form of the at least one cannabis-based additive, the form including at least one of a liquid, a powder, a crystallization, a resin, and combinations thereof.

10. A system for adding consistent amounts of cannabis concentrate in a manufacturing process, the system comprising:
memory that stores information in a database regarding a plurality of different food products, the stored information regarding each food product including a limit on cannabis-based additives;
a communication network interface that receives a request for information regarding a specified food product, the request sent from a manufacturing system over a communication network;
a processor that executes an analytics module stored in memory, wherein execution of the analytics module identifies a quantity of at least one cannabis-based additive that falls within the limit associated with the specified food product based on the stored information;
wherein the communication network interface transmits the identified quantity of the at least one cannabis-based additive from the analytics module to the manufacturing system via the communications network, wherein the manufacturing system is instructed to add the identified quantity of cannabis-based additive to the food product; and
a quality control module executable to evaluate a final composition of the food product to confirm an amount of the cannabis concentrate in the final composition of the food product.

11. The system of claim 10, wherein the limit on cannabis-based additives includes a permitted range of variability.

12. The system of claim 10, wherein the analytics module identifies the quantity of the at least one cannabis-based additive by retrieving the stored information regarding the specified food product from the database, and applying the associated limit to a serving size of the specified food product.

13. The system of claim 10, wherein the stored information regarding to the specified food product includes at least one of serving size, a number of servings per production batch, and a type of food product.

14. The system of claim 10, wherein the request specifies at least one of serving size and number of servings of the specified food product.

15. The system of claim 10, wherein the manufacturing system is instructed to add the cannabis-based additive to the specified food product by:
measuring out the identified quantity of the at least one cannabis-based additive using a measurement device; and
applying the measured quantity of cannabis-based additive to the specified food product via an applicator.

16. The system of claim 10, wherein the quality control module evaluates a final composition of the food product by:
measuring the amount of the cannabis-based additive that is detected in the final composition of the food product using a sensor;
comparing the measured amount of detected cannabis-based additive to the identified quantity; and
determining that the comparison between the measured amount and the identified quantity falls within the limit associated with the specified food product.

17. The system of claim 10, further comprising a removal device that removes the food product from a production line of the manufacturing system based on the amount of cannabis-based additive exceeding the limit associated with the specified food product.

18. The system of claim 10, wherein the analytics module is further executable to select a form of the at least one cannabis-based additive, the form including at least one of a liquid, a powder, a crystallization, a resin, and combinations thereof.

19. A non-transitory, computer-readable storage medium, having embodied thereof a program executable by a processor to perform a method for adding a quantity of cannabis concentrate to a food product, the method comprising:
storing information in a database in memory regarding a plurality of different food products, the stored information regarding each food product including a limit on cannabis-based additives;
receiving a request for information regarding a specified food product, the request sent from a manufacturing system over a communication network to an analytics platform server;
identifying a quantity of at least one cannabis-based additive that falls within the limit associated with the specified food product based on the stored information;
transmitting the identified quantity of the at least one cannabis-based additive from the analytics module to the manufacturing system via the communications network, wherein the manufacturing system is instructed to add the identified quantity of cannabis-based additive to the food product; and
evaluating a final composition of the food product to confirm an amount of the cannabis concentrate in the final composition of the food product.
